(19) United States
(12) Patent Application Publication
(10) Pub. No.: US 2018/0207392 A1
(43) Pub. Date: Jul. 26, 2018

(54) Sleep-aiding Apparatus

(71) Applicant: Nan Wang, Hong Kong (CN)

(72) Inventor: Nan Wang, Hong Kong (CN)

(21) Appl. No.: 15/411,393

(22) Filed: Jan. 20, 2017

(51) Int. Cl. A61M 21/02; A61N 5/06

(52) U.S. Cl. CPC A61M 21/02; A61N 5/0618; A61M 2021/0044; A61N 2005/0663; A61N 2005/0652

(57) ABSTRACT

An embodiment of the utility model disclosed a sleep-aiding apparatus, which comprises a surface case of the body and a bottom case of the body fastened with each other. The top of the surface case of the body is provided with a primary light-emitting spot, a switch button connected with the primary light-emitting spot, and a secondary light-emitting ring provided around the switch button; the bottom case of the body is provided with a PCB circuit board which is connected with the light-emitting spot, the switch button and the secondary light-emitting ring on the surface case of the body; LED lamp beads acting as the carrier are provided in the primary light-emitting spot and the secondary light-emitting ring, and light guide surfaces capable of releasing red lights having optical wavelengths ranging from 4 to 20 microns are provided outside the primary light-emitting spot and the secondary light-emitting ring; innovation of the technical solution has broken through the restriction on the energy products such as necessity of being attached to a body and used within a close range so as to be influenced by the frequency vibration, the use of red lights of 630 nm as the carrier for conducting the energy can make use of the energy of the optical wavelength ranging from 4-20 micrometers more efficiently, conveniently and flexibly.

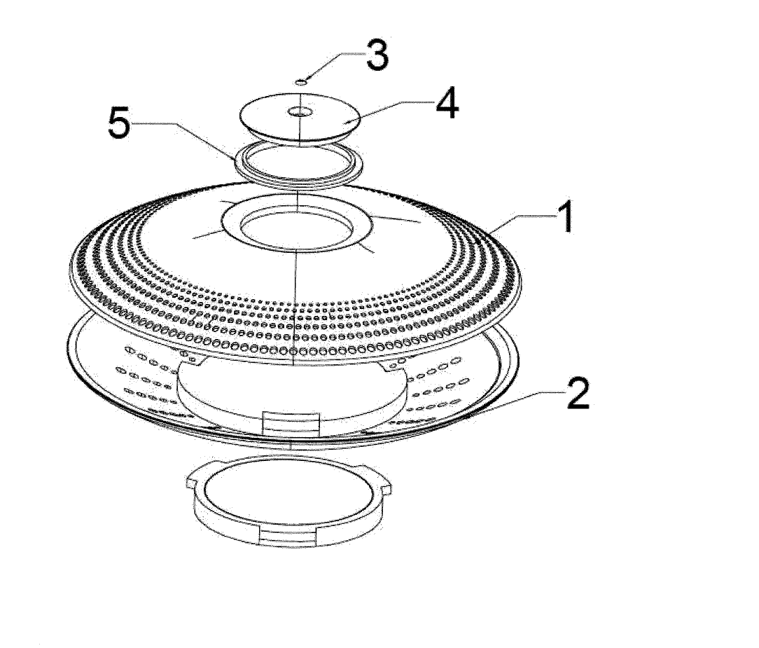

### TECHNICAL FIELD

The utility model relates to the mechanical field, in particular to a sleep-aiding apparatus.

### BACKGROUND ART

The far-infrared energy products at present generally have to be capable of being attached to a body, and positive impact brought by frequency vibration will be exerted only when the human body is in a close-range contact of from 1 to 5 mm with the products themselves. For example, the Chinese patent CN130519601A disclosed a use of a far-infrared energy waterbed, which is implemented by adding the water containing far-infrared energy to the heat preservation mattress to allow physiotherapy of the human body while they are sleeping. Although this apparatus has certain effects, it is only used in a close range within the mattress, thus either the scope of use or the flexibility thereof is restricted.

### SUMMARY OF THE INVENTION

The technical problem to be solved by the embodiment of the utility model is to provide a sleep-aiding apparatus which is mainly operated to transmit a vibration frequency with a red wavelength having optical waves ranging from 4 to 20 microns to the outside of the red light irradiation coverage, so as to improve sleep.

The sleep-aiding apparatus, comprising a surface case of the body and a bottom case of the body fastened with each other, the top of the surface case of the body is provided with a primary light-emitting spot, a switch button connected with the primary light-emitting spot, and a secondary light-emitting ring provided around the switch button; the bottom case of the body is provided with a PCB circuit board which is connected with the light-emitting spot, the switch button and the secondary light-emitting ring on the surface case of the body; LED lamp beads acting as the carrier are provided in the primary light-emitting spot and the secondary light-emitting ring, and light guide surfaces capable of releasing red lights having optical wavelengths ranging from 4 to 20 microns are provided outside the primary light-emitting spot and the secondary light-emitting ring.

2835 LED light beads with wavelengths of 630 micrometers are provided in the primary light-emitting spot, with light emitted at a frequency of 20 times per minute, 1500 ms each time, and a quench time of 1500 ms after the lights are emitted.

A 0805 LED acting as a light source is provided in the secondary luminescent ring, and a high-gloss finish transparent polymethyl methacrylate rubber ring is further arranged for guiding lights.

An anti-skid pad and a wall-hung base are provided below the bottom case of the body. The surface case of the body is made from aluminum alloy by CNC processing, and the bottom case of the body is made by injection molding of ABS rubber.

Implementation of the embodiment of the utility model has the following beneficial effects: an embodiment of the utility model is about a sleep-aiding apparatus, in which visible red lights of 630 nanometers are adopted by the apparatus as the carrier, and the vibration frequency with a wavelength ranging from 4 to 20 microns is transmitted to the red light irradiation coverage so as to form an electromagnetic energy field, so that the human body within this field range can obtain a far-infrared ray energy, the far-infrared ray vibration frequency having a wavelength ranging from 4 to 20 microns is close to the vibration frequency of the cellular elements in the human body, the vibration frequency can effectively be involved in resonance effect with the cells in the body after they penetrate into the body to raise the subcutaneous deep temperature, dilate blood vessels, and improve microvascular circulation, so as to activate tissue cells, and relieve a variety of diseases or discomforts caused by microcirculation disorders, innovation of the invention has broken through the restriction on the energy products such as necessity of being attached to a body and used within a close range so as to be influenced by the frequency vibration, the use of red lights of 630 nm as the carrier for conducting the energy can make use of the energy of the optical wavelength ranging from 4 to 20 micrometers more efficiently, conveniently and flexibly.

### BRIEF DESCRIPTION OF THE DRAWINGS

The sole FIGURE is a schematic structural view of an embodiment of the present invention.

### DETAILED DESCRIPTION OF THE INVENTION

To make the purpose, technical solution and advantages of the present utility model more apparent, the present utility model is further illustrated in conjunction with the accompany drawings.

An embodiment of the present utility model is about a sleep-aiding apparatus, comprising a surface case of the body 1 and a bottom case of the body 1 fastened with each other, the top of the surface case of the body 1 is provided with a primary light-emitting spot 3, a switch button 4 connected with the primary light-emitting spot 3, and a secondary light-emitting ring 5 provided around the switch button; the bottom case of the body is provided with a PCB circuit board which is connected with the light-emitting spot 3, the switch button 4 and the secondary light-emitting ring 5 on the surface case of the body; LED lamp beads acting as the carrier are provided in the primary light-emitting spot 3 and the secondary light-emitting ring 5, and light guide surfaces capable of releasing red lights having optical wavelengths ranging from 4 to 20 microns are provided outside the primary light-emitting spot and the secondary light-emitting ring; 2835 LED light beads with wavelengths of 630 micrometers are provided in the primary light-emitting spot, with light emitted at a frequency of 20 times per minute, 1500 ms each time, and a quench time of 1500 ms after the lights are emitted; a 0805 LED acting as a light source is provided in the secondary luminescent ring, and a high-gloss finish transparent polymethyl methacrylate rubber ring is further arranged for guiding lights; the PCB circuit board is welded with a music player, and a sound volume button and a brightness button respectively connected with the PCB circuit board are provided on either side of the bottom case of the body for adjusting the sound volume and the brightness, the brightness is divided into two levels, and an anti-skid pad and a wall-hung base are provided below the bottom case of the body. The surface case of the body is made from 6061 T4 aluminum alloy by CNC processing, and the bottom case of the body is made by injection molding of ABS rubber.

An embodiment of the utility model is about a sleep-aiding apparatus, in which visible red lights of 630 nanometers are adopted by the apparatus as the carrier, and the vibration frequency with a wavelength ranging from 4 to 20 microns is transmitted to the red light irradiation coverage so as to form an electromagnetic energy field, so that the human body within this field range can obtain a far-infrared ray energy, the far-infrared ray vibration frequency having a wavelength ranging from 4 to 20 microns is close to the vibration frequency of the cellular elements in the human body, the vibration frequency can effectively be involved in resonance effect with the cells in the body after they penetrate into the body to raise the subcutaneous deep temperature, dilate blood vessels, and improve microvascular circulation, so as to activate tissue cells, and relieve a variety of diseases or discomforts caused by microcirculation disorders, innovation of the invention has broken through the restriction on the energy products such as necessity of being attached to a body and used within a close range so as to be influenced by the frequency vibration, the use of red lights of 630 nm as the carrier for conducting the energy can make use of the energy of the optical wavelength ranging from 4-20 micrometers more efficiently, conveniently and flexibly.

The disclosed is only a more preferable embodiment of the present utility model, which of course cannot be construed as limiting the protection scope of the present utility model, but is still within the coverage of the present utility model in accordance with the equivalent variations made for the claims of the present utility model.

1. A sleep-aiding apparatus, characterized by comprising a surface case of the body and a bottom case of the body fastened with each other, the top of the surface case of the body is provided with a primary light-emitting spot, a switch button connected with the primary light-emitting spot, and a secondary light-emitting ring provided around the switch button; the bottom case of the body is provided with a PCB circuit board which is connected with the light-emitting spot, the switch button and the secondary light-emitting ring on the surface case of the body; LED lamp beads acting as the carrier are provided in the primary light-emitting spot and the secondary light-emitting ring, and light guide surfaces capable of releasing red wavelengths ranging from 4 to 20 microns are provided outside the primary light-emitting spot and the secondary light-emitting ring.

2. The sleep-aiding apparatus according to claim 1, characterized in that 2835 LED light beads with wavelengths of 630 micrometers are provided in the primary light-emitting spot, with light emitted at a frequency of 20 times per minute, 1500 ms each time, and a quench time of 1500 ms after the lights are emitted.

3. The sleep-aiding apparatus according to claim 1, characterized in that a 0805 LED acting as a light source is provided in the secondary luminescent ring, and a high-gloss finish transparent polymethyl methacrylate rubber ring is further arranged for guiding lights.

4. The sleep-aiding apparatus according to claim 1, characterized in that an anti-skid pad and a wall-hung base are provided below the bottom case of the body.

5. The sleep-aiding apparatus according to claim 1, characterized in that the surface case of the body is made from aluminum alloy by CNC processing, and the bottom case of the body is made by injection molding of ABS rubber.
